(19) United States
(12) Patent Application Publication
(10) Pub. No.: US 2025/0177253 A1
(43) Pub. Date: Jun. 5, 2025

(54) Holding Device for Medical Containers With Plates of Different Elevations

(71) Applicant: Becton Dickinson France, Le Pont-de-Claix (FR)

(72) Inventors: Simon Rempfer, St Hilaire de la Cote (FR); Mathilde Paulet, Grenoble (FR); Jérémie Vaxelaire, Ambilly (FR); Ferdinand Lavigne, Seyssinet-Pariset (FR); Anaïs Eymery, Saint-Georges-de-Commiers (FR)

(21) Appl. No.: 18/840,515

(22) PCT Filed: Feb. 23, 2023
(86) PCT No.: PCT/EP2023/054554

§ 371 Date: Aug. 22, 2024

(30) Foreign Application Priority Data
Feb. 23, 2022 (EP) 22305202.8

(51) Int. Cl. A61J 1/16

(52) U.S. Cl. CPC A61J 1/16

(57) ABSTRACT

A holding device configured to support medical containers includes a supporting plate having a first surface, a second surface, a peripheral edge between the first surface and the second surface, and a plurality of openings through the supporting plate configured to receive the medical containers. The surfaces of the supporting plate include a peripheral lower portion extending inward from the peripheral edge; at least one upper portion enclosed by the peripheral lower portion; and at least one upwardly angled portion extending between the lower portion and the upper portion. The holding device further includes a plurality of chimneys protruding from the first surface and/or the second surface of the supporting plate at least partially enclosing the plurality of openings, such that the plurality of chimneys guide insertion of the medical containers into the plurality of openings.

### CROSS-REFERENCE TO RELATED APPLICATION

The present application claims priority to European Application No. 22305202.8 entitled “Holding Device for Medical Containers with Plates of Different Elevations” filed Feb. 23, 2022, the entire disclosure of which is hereby incorporated by reference in its' entirety.

### BACKGROUND OF THE INVENTION

### Field of the Invention

The present disclosure relates generally to holding devices for holding medical containers, such as cartridges, barrels, prefilled-syringes, stoppers, and/or pre-fillable syringes, in an upright position and, in particular, to holding devices including supporting structures, such as plates or trays, configured to be attached to containers or tubs for containing the medical containers.

### Description of Related Art

Medical containers, such as cartridges, barrels, prefilled-syringes, stoppers, and/or pre-fillable syringes, often need to be transported from one site to another site during or after manufacturing. For example, medical containers may be manufactured at a first site and then transported to a second site, where the medical containers are filled with a medical fluid. As used herein, a “medical fluid” can refer to a medication or another therapeutic agent used for treatment of chronic or acute conditions, as are known in the art. Exemplary therapeutic agents can include, for example, drugs, chemicals, biological, or biochemical substances that, when delivered in a therapeutically effective amount to the patient, achieve a desired therapeutic effect. In other examples, the medical containers can be manufactured and filled at a same first site and then transported to a second site for storage.

During transport, the medical containers can be held by a holding device, such as a tray, plate, and/or nest. A nest can be a plate-shaped tray configured to support multiple medical containers in an upright orientation. Nests can comprise multiple through-holes or openings aligned according to predetermined rows, with each through-hole being configured to receive one medical container. When inserted into the through-hole, the medical container is held in the upright orientation in a direction substantially orthogonal to the nest. The nest is usually placed inside a box-shaped tub with an open top, which can be sealed by a sealing cover. Removal of the medical containers from this tub can require peeling off the sealing cover, removing the nest holding the medical containers from the tub, and removing the medical containers from the nest by axially sliding the medical containers relative to the nest. Exemplary nests and tubs configured to contain medical containers are disclosed, for example, in: U.S. Pat. No. 10,023,358 (“the '358 patent”), entitled “Packaging for containers;” U.S. Patent No. 10,143,793 (“the '793 patent”), entitled “Tray for positioning elongated objects, in particular syringe bodies or syringes;” and U.S. Patent Appl. Pub. No. 2021/0236715A1 (“the '715 publication”), entitled “Holding Device Configured to Support a Plurality of Medical Containers Such as Syringes,” which are incorporated herein by reference in their entireties.

In many cases, the medical containers are filled with the medical fluid using an automated filling machine before the medical containers are removed from the nest and individually packaged. The nests can deform during filling due to forces exerted on the medical containers and/or nest by the automated filing machine. For example, when handled by the filling machine, the nests may bend or warp due to the weight of multiple medical containers held by the nest. As a result of deformation of the nest or trays, medical containers supported by the deformed trays may not be retained in the upright orientation, which may lead to some inaccuracy during the filling process and/or during a stoppering process. Deformation of the nests or trays may also cause problems during conveying of the filled nests or trays to different locations.

For these reasons, there is a need for nest and tray designs with increased stiffness in order to reduce deflection of the nest or tray during transport and filling. Avoiding deformation can improve accuracy of the filling process by avoiding misalignment between the medical containers contained by the tray or nest and the automated filling machines. The holding devices of the present disclosure are intended to provide such increased stiffness and to resist deflection or deformation under weight of multiple medical containers contained by the tray or nest.

### SUMMARY OF THE INVENTION

According to an aspect of the disclosure, a holding device configured to support medical containers includes a supporting plate having a first surface, a second surface, a peripheral edge between the first surface and the second surface, and a plurality of openings through the supporting plate configured to receive the medical containers. The surfaces of the supporting plate include a peripheral lower portion extending inward from the peripheral edge; at least one upper portion enclosed by the peripheral lower portion; and at least one upwardly angled portion extending between the lower portion and the upper portion. The holding device further includes a plurality of chimneys protruding from the first surface and/or the second surface of the supporting plate at least partially enclosing the plurality of openings, such that the plurality of chimneys guide insertion of the medical containers into the plurality of opening.

According to another aspect of the disclosure, an assembly configured to support a plurality of medical containers includes a tub and the previously described holding device contained in the tub.

According to another aspect of the present disclosure, a method of manufacture for the previously described holding device includes injection molding or three-dimensional printing the support plate and the plurality of chimneys of the holding device as a single part by a single injection molding or three-dimensional printing process.

Non-limiting illustrative examples of embodiments of the present disclosure will now be described in the following numbered clauses:

Clause 1: A holding device configured to support medical containers, comprising: a supporting plate comprising a first surface, a second surface, a peripheral edge between the first surface and the second surface, and a plurality of openings through the supporting plate configured to receive the medical containers, wherein the surfaces of the supporting plate comprise: a peripheral lower portion extending inward from the peripheral edge; at least one upper portion enclosed by the peripheral lower portion; and at least one upwardly angled portion extending between the lower portion and the upper portion; and a plurality of chimneys protruding from the first surface and/or the second surface of the supporting plate at least partially enclosing the plurality of openings, such that the plurality of chimneys guide insertion of the medical containers into the plurality of openings.

Clause 2: The holding device of clause 1, wherein the plurality of openings are arranged on the supporting plate as a plurality of rows and a plurality of columns.

Clause 3: The holding device of clause 2, wherein openings of at least one row of the plurality of rows and openings of at least one column of the plurality of columns are equidistantly spaced.

Clause 4: The holding device of clause 2 or clause 3, wherein the plurality of openings are arranged on the supporting plate as at least five columns and at least five rows.

Clause 5: The holding device of any of clauses 2-4, wherein openings of adjacent rows are aligned parallel to a transverse axis of the supporting plate and openings of adjacent columns are aligned parallel to a longitudinal axis of the supporting plate orthogonal to the transverse axis.

Clause 6: The holding device of any of clauses 2-5, wherein the supporting plate comprises more columns than rows.

Clause 7: The holding device of any of clauses 1-6, wherein, when fully loaded with the medical containers, deflection of the supporting plate is less than 2.5 mm in a vertical direction.

Clause 8: The holding device of any of clauses 1-7, wherein the supporting plate and the plurality of chimneys is made from a thermoplastic polymer, such as polypropylene.

Clause 9: The holding device of any of clauses 1-8, wherein the supporting plate has a substantially constant thickness.

Clause 10: The holding device of any of clauses 1-9, wherein the supporting plate and the plurality of chimneys are integrally formed, such as by injection molding or three-dimensional printing.

Clause 11: The holding device of any of clauses 1-10, wherein the supporting plate comprises fewer than 100 openings.

Clause 12: The holding device of any of clauses 1-11, wherein the supporting plate comprises fewer than 50 openings.

Clause 13: The holding device of any of clauses 1-12, wherein the peripheral lower portion intersects at least the outer chimneys of the plurality of chimneys.

Clause 14: The holding device of any of clauses 1-13, wherein the peripheral lower portion of the surfaces is flat.

Clause 15: The holding device of any of clauses 1-14, wherein the upper portion of the surfaces is flat.

Clause 16: The holding device of any of clauses 1-15, wherein the upper portion is elevated relative to the lower portion by a vertical distance of about 2.0 mm to about 14.5 mm.

Clause 17: The holding device of any of clauses 1-16, wherein the at least one upper portion intersects at least one of the plurality of chimneys.

Clause 18: The holding device of any of clauses 1-17, wherein the at least one upper portion of the surfaces is a convex surface or a concave surface.

Clause 19: The holding device of any of clauses 1-18, wherein the angled portion intersects at least one of the plurality of chimneys.

Clause 20: The holding device of any of clauses 1-19, wherein the angled portion is substantially vertical extending substantially orthogonally from the lower portion.

Clause 21: The holding device of clause 20, wherein the vertical angled portion has a draft angle of about 0.1 degree to about 4.0 degrees.

Clause 22: The holding device of any of clauses 1-21, wherein the vertical angled portion intersects at least two adjacent chimneys of the plurality of chimneys.

Clause 23: The holding device of any of clauses 1-22, wherein the angled portion comprises alternating inwardly angled segments, which are angled towards a center of the first surface and/or the second surface of the supporting plate, and outwardly angled segments, which are angled away from the center of the first surface and/or the second surface of the supporting plate.

Clause 24: The holding device of any of clauses 1-23, wherein the peripheral edge comprises at least one cutout extending inward from other portions of the peripheral edge.

Clause 25: The holding device of clause 24, wherein the at least one cutout comprises a first cutout on a first side of the supporting plate and a second cutout on a second side of the supporting plate opposite the first side, and wherein the first and second cutouts are aligned along a first axis of the supporting plate.

Clause 26: The holding device of clause 24 or clause 25, further comprising a curved wall extending from the first surface and/or the second surface and about at least a portion of the at least one cutout.

Clause 27: The holding device of any of clauses 1-26, wherein the plurality of chimneys comprise a tubular wall comprising a cylindrical inner surface having a diameter substantially the same as the diameter of the opening enclosed by the chimney and a cylindrical outer surface opposite the inner surface.

Clause 28: The holding device of clause 27, wherein the plurality of chimneys further comprise at least two axial slits separating the tubular wall into at least two guiding tabs.

Clause 29: The holding device of any of clauses 1-28, the plurality of openings and the plurality of chimneys are configured to receive barrels of syringes or cartridges containing a fluid volume of less than 50 mL.

Clause 30: The holding device of any of clauses 1-29, wherein the surfaces of the supporting plate further comprise a second lower portion at least partially enclosed by the upper portion and a downwardly angled portion extending between the upper portion and the second lower portion.

Clause 31: The holding device of clause 30, wherein the second lower portion and the downwardly angled portion each intersect at least one of the chimneys of the plurality of chimneys.

Clause 32: The holding device of any of clauses 1-31, wherein the at least one upper portion comprises a first upper portion and a second upper portion, which is separated from the first upper portion by the lower portion.

Clause 33: The holding device of clause 32, wherein the first upper portion and the second upper portion each intersect at least one of the plurality of chimneys.

Clause 34: The holding device of clause 33, wherein the surfaces of the supporting plate further comprise a second upper portion at least partially enclosed by the second lower portion and a second upwardly angled surface extending between the second lower portion and the second upper portion.

Clause 35: An assembly configured to support a plurality of medical containers, the assembly comprising: a tub; and the holding device of any of clauses 1-34 contained in the tub.

Clause 36: A method of manufacture for the holding device of any of clauses 1-34, the method comprising injection molding or three-dimensional printing the support plate and the plurality of chimneys by a single injection molding or three-dimensional printing process.

### BRIEF DESCRIPTION OF THE DRAWINGS

FIG. 1A is a top perspective view of a holding device configured to support medical containers, according to an aspect of the present disclosure.

FIG. 1B is a top view of the tray of FIG. 1A.

FIG. 1C is a cross-sectional view of the tray of FIG. 1A.

FIG. 2A is a top perspective view of another example of a holding device configured to support medical containers, according to an aspect of the present disclosure.

FIG. 2B is a bottom perspective view of the holding device of FIG. 2A.

FIG. 2C is a top view of the holding device of FIG. 2A.

FIG. 2D is a cross-sectional view of the tray of FIG. 2A.

FIG. 3A is a top view of another example of a holding device configured to support medical containers, according to an aspect of the present disclosure.

FIG. 3B is a cross-sectional view of the tray of FIG. 3A.

FIG. 4A is a perspective view of another example of a holding device configured to support medical containers, according to an aspect of the present disclosure.

FIG. 4B is a top view of the tray of FIG. 4A.

FIGS. 5A-5G are schematic drawings showing top views of additional examples of holding devices configured to support medical containers, in which cross-hatching represents raised or elevated surfaces of the holding devices, according to aspects of the present disclosure.

FIGS. 6A-6C are schematic drawings of top views of additional examples of holding devices configured to support medical containers including segmented vertical walls and in which cross-hatching represents raised or elevated surfaces, according to aspects of the present disclosure.

FIGS. 7A and 7B are cross-sectional views of additional examples of holding devices configured to support medical containers, according to aspects of the present disclosure.

FIG. 8A is a top perspective view of another example of a holding device configured to support medical containers, according to an aspect of the present disclosure.

FIG. 8B is a top view of the holding device of FIG. 8A.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, “transverse”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to holding devices 10 comprising openings or cavities for retaining multiple medical containers, such as multiple cartridges, barrels, stoppers, prefilled-syringes, and/or pre-fillable syringes, in an upright position. In particular, the holding devices 10 can be configured to concurrently support multiple medical containers in the upright position during packaging, storing, transporting, and/or handling of the multiple medical containers with an automated filling machine.

A medical container, such as a syringe, is generally an elongate structure that comprises a hollow elongated barrel defining a reservoir for containing the medical fluid, a stopper located inside the barrel, and a plunger rod to move the stopper through the barrel to expel the medical fluid through a tip, fluid port, nozzle, or needle cannula at an end of the barrel. The reservoirs of the medical containers held by the holding devices 10 of the present disclosure can contain less than about 50 mL, or preferably from about 1 mL to 10 mL, of the medical fluid. The medical containers can also include a flange at an end of the barrel, which can provide a surface for positioning a user's fingers (e.g., the index and the middle fingers), while the plunger rod is activated with the thumb. The holding device 10 can be configured to retain fewer medical containers than most currently available trays or supporting plates. For example, the holding devices 10 can be configured to contain 160 or fewer medical containers, preferably 100 or fewer medical containers, or more preferably, 50 or fewer medical containers. For example, the holding device 10 can be configured to contain 42 medical containers.

In some examples, the holding devices 10 can include plates or surfaces of different elevations in order to increase stiffness of the holding device 10 and to resist deformation of the holding device 10. In particular, the holding devices 10 can be configured to resist deformation when the holding devices 10 are used with the automated filling machine, such that downward deflection of the holding device 10 is 2.5 mm or less during filling, when the holding device 10 is substantially fully loaded with filled medical containers. Specifically, the holdings devices 10 can be configured to deflect by about 2.5 mm or less, for a holding device 10 having a thickness of 1.27 mm comprising 42 cavities loaded with filled 10 mL medical containers, as determined by finite element analysis (FEA). By contrast, a conventional holding device or plate with a plate thickness of 1.27 mm and including 42 cavities, but without surfaces of different elevations, was found by FEA to deflect by over 20 mm.

As previously described, the holding devices 10 of the present disclosure are configured to be disposed in a container, such as a tub. Exemplary tubs are described in the '358 patent, the '793 patent, and the '715 publication referred to previously. For example, the holding devices 10 of the present disclosure can be disposed across the open top of a tub to cover an interior of the tub. The medical containers can be inserted through through-holes or openings of the holding devices 10, such that distal portions of the medical containers (i.e., portions of the medical containers that are farthest away from the portion of the device or container grasped or manipulated by a user) are in the interior of the tub and a sidewall of the medical container is supported by the through-hold or opening of the holding devices 10.

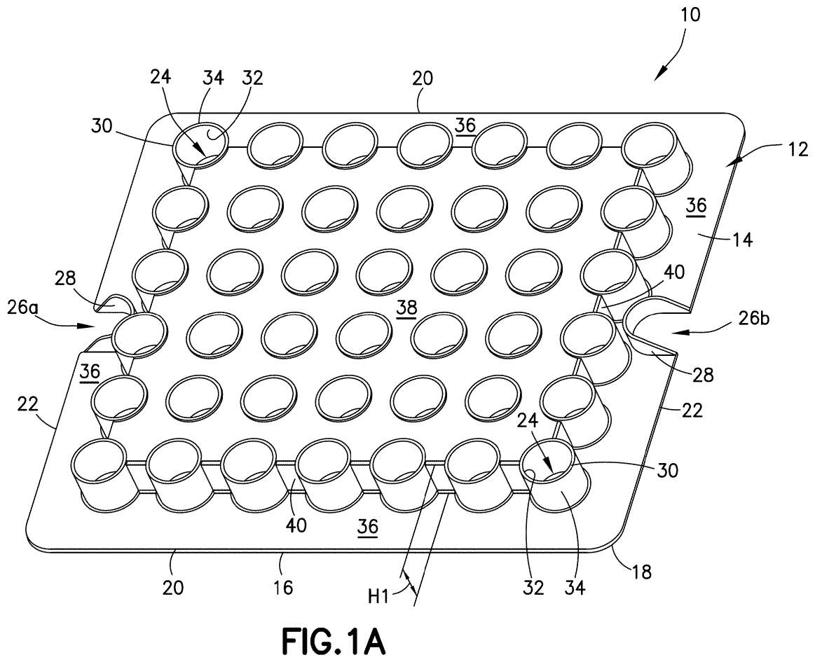

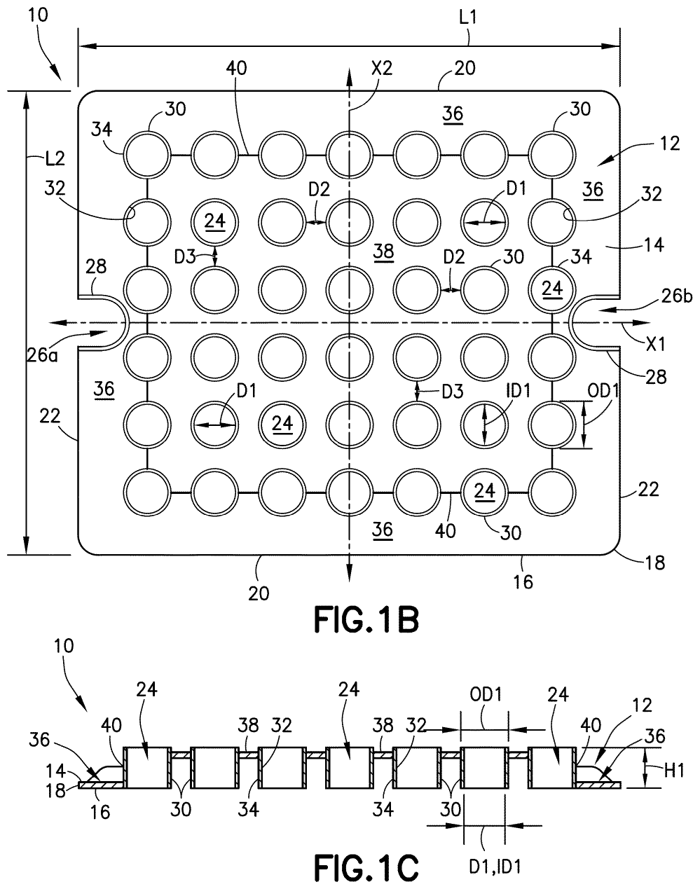

FIGS. 1A-1C illustrate an exemplary holding device 10 configured to support medical containers including features of the present disclosure. The holding device 10 comprises a supporting plate 12 having a first or upper surface 14, a second or lower surface 16, and a peripheral edge 18 extending about the supporting plate 12 between the upper surface 14 and the lower surface 16. In some examples, the supporting plate 12 is substantially rectangular having longitudinal sides 20 and transverse sides 22. As used herein, the “longitudinal sides” refer to longer sides 20 of the supporting plate 12 having a length L1 (shown in FIG. 1B) and the “transverse sides” refer to shorter sides 22 of the supporting plate 12 having a length L2, which is shorter than the length L1. The supporting plate 12 may also be square, in which case the longitudinal sides 20 and the transverse sides 22 are the same length (i.e., Ll is equal to L2). As shown in FIG. 1B, the substantially rectangular supporting plate 12 defines a first axis X1 (also referred to as a longitudinal, horizontal, or x axis) extending parallel to the longitudinal sides 20 of the supporting plate 12 and a second axis X2 (also referred to as a transverse, latitudinal, vertical, or y axis) extending parallel to the transverse sides 22 of the supporting plate 12.

Dimensions of the supporting plate 12 will be determined by those skilled in the art based on the number of medical containers that can be supported by the holding device 10, dimensions of the medical containers, and/or dimensions of the tub or container to which the holding device 10 is mounted. For example, the length L1 can be from about 228.8 mm to about 231.2 mm and the length L2 can be from about 197.1 mm to about 199.5 mm. A thickness of the supporting plate can be from about 1.0 mm to about 2.0 mm or, preferably from about 1.3 mm to about 1.7 mm. In some examples, dimensions of the supporting plate 12 can be determined based on published standards for pre-filled syringe packaging dimensions (e.g., ISO 11040-7:2015(E): Packaging systems for sterilized sub-assembled syringes ready for filling).

The supporting plate 12 further comprises multiple openings 24 through the supporting plate 12 configured to receive the medical containers. The openings 24 are sized to support to medical containers in an upright position. For example, the openings 24 can be wide enough so that a sidewall of the medical container passes through the openings 24, while preventing a flange or another protruding portion of the medical container from passing through the openings 24. Accordingly, when the medical container is inserted through one of the openings 24, the flange of the medical container may rest against the upper surface 14 of the supporting plate 12, thereby holding the medical container in the upright position. The dimensions of the openings 24 are generally dependent upon a size of the medical container intended to be used with the holding device 10. For example, an inner diameter D1 (shown in FIGS. 1B and 1C) of the openings 24 can be from about 18.06 mm to about 19.06 mm for a supporting plate 12 comprising forty-two openings 24. For a supporting plate 12 comprising one-hundred openings 24, the inner diameter of the openings 24 can be from about 12.1 mm to about 12.35 mm.

In some examples, the supporting plate 12 can include 160 or fewer openings 24 or, preferably fifty or fewer openings 24. For example, the supporting plate 12 can comprise forty-two openings 24. The openings 24 can be arranged on the supporting plate 12 as rows and columns. For example, the supporting plate 12 can include at least five rows and at least five columns. In order for the supporting plate 12 to have a rectangular shape, in some examples, the supporting plate 12 includes a greater number of columns than rows. As shown in FIGS. 1A and 1B, in one example, the supporting plate 12 includes seven columns, which extend parallel to the transverse or second axis X2 and six rows, which extend parallel to the longitudinal or first axis X1 of the supporting plate 12. The openings 24 of each row and the openings 24 of each column can be equidistantly spaced. For example, an opening 24 of a row can be spaced apart from an adjacent opening 24 of the same row by a distance D2 (shown in FIGS. 1B and 1C) of from about 8.9 mm to about 11.11 mm. Similarly, an opening 24 of a column can be spaced apart from an adjacent opening 24 of the same column by a distance D3 (shown in FIGS. 1B and 1C) of about 8.76 mm to about 11.26 mm.

In some examples, openings 24 of the rows and columns are aligned. For example, openings 24 of adjacent rows can be aligned along virtual lines V2 extending parallel to the transverse or second axis X2. In particular, for a supporting plate 12 with seven columns and six rows, as shown in FIGS. 1A and 1B, each row includes seven openings 24, one opening 24 in each column. In other examples, openings 24 of the rows and/or columns can be offset. For example, each row can include four openings 24. However, the rows can be offset from adjacent rows, such that the supporting plate 12 includes seven columns.

The supporting plate 12 can also include cutouts 26a, 26b extending inwardly from other portions of the peripheral edge 18. For example, as shown in FIGS. 1A and 1B, the supporting plate 12 includes a first cutout 26a on one of the transverse sides 22 of the supporting plate 12 and a second cutout 26b on a second transverse side 22 of the supporting plate 12. The first and second cutouts 26a, 26b can be aligned along the longitudinal or first axis X1 of the supporting plate 12. In some examples, the cutouts 26a, 26b are a u-shaped gap or opening extending inwardly from the peripheral edge 18 of the supporting plate 12. The holding device 10 can also include a curved wall 28 extending from the upper surface 14 and/or the lower surface 16 of the supporting plate 12 and about at least a portion of the first cutout 26a and/or the second cutout 26b.

The holding device 10 further comprises tubular walls, members, or ridges (referred to herein as chimneys 30) protruding from the upper surface 14 and/or the lower surface 16 of the supporting plate 12. Each chimney 30 encloses or partially encloses one of the openings 24 and is positioned to guide insertion of the medical containers into the opening 24. The chimneys 30 can include a tubular wall comprising a cylindrical inner surface 32 having a diameter ID1 (shown in FIGS. 1B and 1C) substantially the same as the diameter D1 of the opening 24 enclosed or partially enclosed by the chimney 30 and a cylindrical outer surface 34 opposite the inner surface 32 having an outer diameter OD1. For example, for the supporting plate 12 comprising 42 chimneys 30, the inner diameter ID1 can be about 18.06 mm to about 19.06 mm and the outer diameter OD1 can be about 18.96 mm to about 21.06 mm, meaning that a thickness of the chimney 30 is about 0.9 mm to about 2.0 mm. A height of the chimneys 30 (e.g., a distance between the upper surface 14 of the supporting plate 12 and a top of the chimney 30) can be about 17.23 mm to about 17.73 mm.

As shown in FIGS. 1A and 1B, the chimneys 30 are tubular structures including the cylindrical inner surface 32 and the cylindrical outer surface 34. In other examples, the chimneys 30 can comprise axial slits or gaps extending through the chimneys 30, which separate the tubular wall of the chimney 30 into distinct segments or tabs. For example, a chimney 30 can include two axial slits (not shown), which separate the tubular wall into arcuate guide segments or guide tabs, similar to the slits and guide tabs shown in the '715 publication referred to previously.

As previously described, the holding device 10 includes structures for increasing stiffness of the supporting plate 12 and for resisting deflection or deformation when the holding device 10 is fully filled with medical containers. In particular, the holding device 10 and supporting plate 12 are configured to ensure that downward deflection of the holding device 10, when filled with medical containers, is 2.5 mm or less. In order to increase stiffness of the supporting plate 12, the surfaces 14, 16 of the supporting plate 12 can include portions of different elevations connected by angled or vertical walls. For example, as shown in FIGS. 1A-1C, the first or upper surface 14 and/or the second or lower surface 16 of the supporting plate 12 can include a peripheral lower portion 36 extending inward from the peripheral edge 18 of the supporting plate 12. The surfaces 14, 16 of the supporting plate 12 also include one or more elevated or upper portions 38 enclosed by the peripheral lower portion 36 and one or more upwardly angled portions 40 extending between the peripheral lower portion 36 and the upper portion 38. The openings 24 and/or chimneys 30 extend through or from all portions 36, 38, 40 of the surfaces 14, 16 of the supporting plate 12. For example, outer openings 24 and outer chimneys 30 (i.e., openings 24 and chimneys 30 proximate to the peripheral edge 18 of the supporting plate 12) can pass through or extend from the peripheral lower portion 36 of the surfaces 14, 16. The outer openings 24 and chimneys 30 can also pass through or extend from the upwardly angled portions 40 of the supporting plate 12. Inner openings 24 and inner chimneys 30 (i.e., openings 24 and chimneys 30 proximate to a center of the supporting plate 12 and enclosed by the outer openings 24 and/or outer chimneys 30) can pass through or extend from the upper portion 38 of the surfaces 14, 16.

The peripheral lower portion 36 of the surfaces 14, 16 refers to a lowest portion of the holding device 10 positioned such that, if the holding device 10 is placed on a flat substrate, the peripheral lower portion 36 of the lower or second surface 16 is in face-to-face contact with the flat substrate. The upwardly angled portion 40 and the upper portion 38 of the surfaces 14, 16 are elevated relative to the peripheral lower portion 36, meaning that, when positioned on the flat substrate, the angled portion 40 of the lower surface 16 and the upper portion 38 of the lower surface 16 are not in face-to-face contact with the flat substrate.

In some examples, the surfaces 14, 16 of the peripheral lower portion 36 are flat, meaning that the peripheral lower portion 36 of the surfaces 14, 16 can rest against or be mounted to portions of the tub or container for securing the holding device 12 to the tub or container. In some examples, the holding device 10 can be configured to rest against and/or be supported by parallel rails (e.g., rails extending parallel to either the longitudinal or first axis X1 or the transverse or second axis X2 of the supporting plate 12). In that case, the parallel rails can be placed under the flat surface of the peripheral lower portion 36 proximate to either the longitudinal sides 20 or the transverse sides 22 of the holding device 10. Desirably, the supporting plate 12 is sufficiently stiff to maintain its shape and to resist deformation or deflection even when only supported by the two parallel rails.

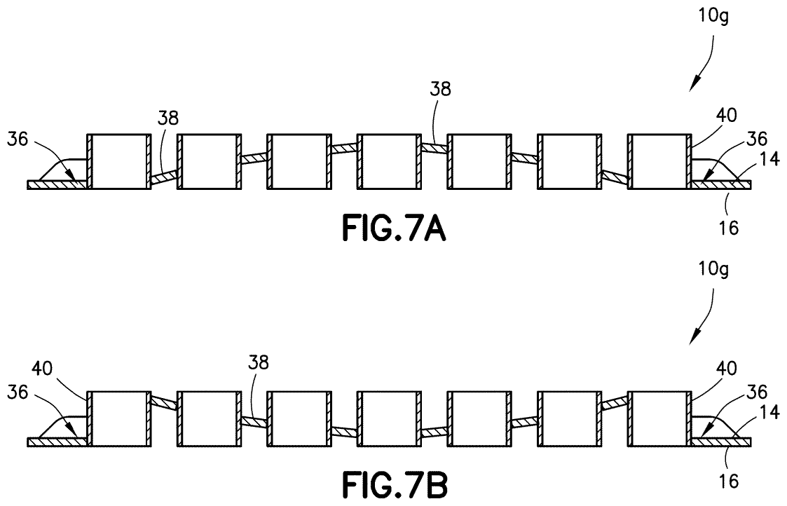

In some examples, the upper portion 38 of the surfaces 14, 16 can be elevated relative to the peripheral lower portion 36 by a height H1 (shown in FIG. 1C), which can be from about 2.9 mm to about 16.5 mm. In some examples, the height HI of the upper portion 38 is about the same as a height of the chimneys 30 (e.g., a height from the first or upper surface 14 of the peripheral lower portion 36 to a top of the chimney 30). In other examples, the height H1 of the upper portion 38 can be less than the height of the chimneys 30. For example, the height H1 of the upper portion 36 can be between 60% and 90% of the full height of the chimneys 30. In some examples, as described in further detail hereinafter, the upper portion 38 of the surfaces 14, 16 is flat. In other examples, as shown in FIGS. 7A and 7B, the upper portion 38 can comprise a convex surface or a concave surface.

The upwardly angled portion 40 of the surfaces 14, 16 extends between the peripheral lower portion 36 and the upper portion 38. As previously described, the angled portion 40 can be connected to and/or extend from the outer chimneys 30 of the holding device 10. For example, the angled portion 40 can intersect and/or extend from at least two adjacent outer chimneys 30 of the holding device 10. In some examples, as shown in FIGS. 1A-1C, the upwardly angled portion 40 comprises a substantially vertical wall extending substantially orthogonally relative to the first or upper surface 14 of the peripheral lower portion 36. In some examples, the substantially vertical wall of the angled portion 40 can have a slight slope, often referred to as a draft angle, to facilitate removing the holding device 10 from a tool or mold following injection molding. For example, the slight slope or draft angle can be about 0.5 degrees or less.

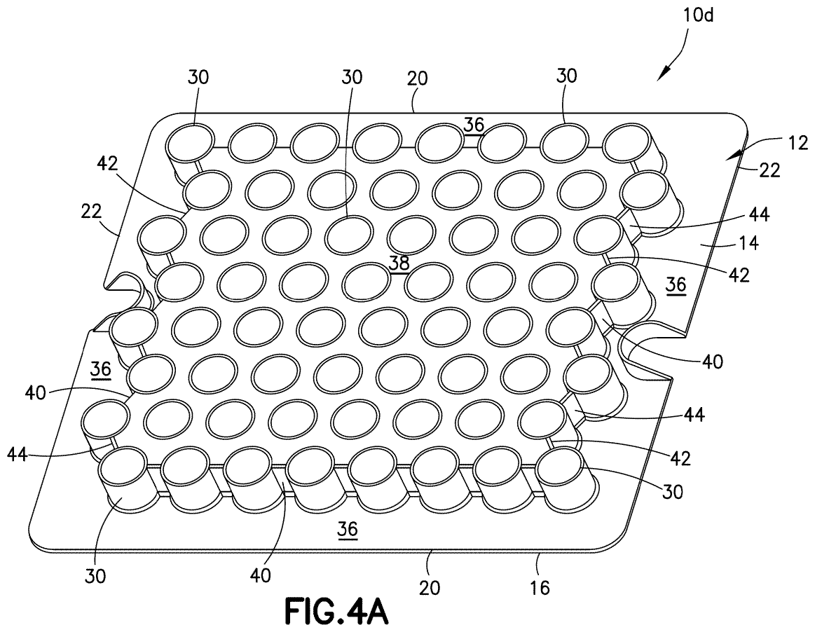

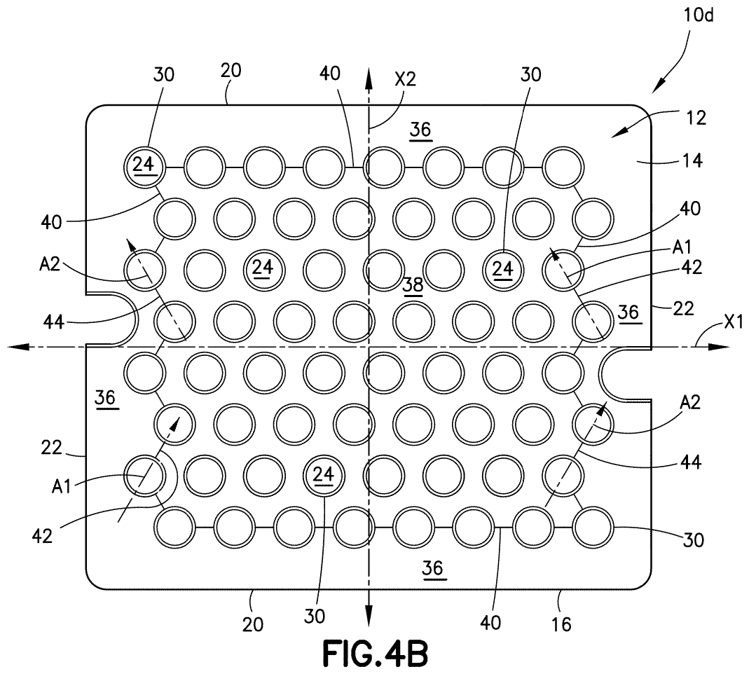

In some examples, as shown in FIGS. 1A-1C, the angled portion extends generally parallel to either the longitudinal or first axis X1 or the transverse or second axis X2 of the supporting plate 12. In other examples, as shown, for example, in FIGS. 4A and 4B, the angled portion 40 is segmented including, for example, alternating inwardly angled segments 42 and outwardly angled segments 44. As described in further detail herein, the inwardly angled segments 42 can be angled towards a center and/or axes X1, X2 of the supporting plate 12. The outwardly angled segments 44 can be angled away from the center or axes X1, X2 of the supporting plate 12.

The surfaces 14, 16 of the supporting plate 12 can include many different arrangements of lower portions 36, angled portions 40, and upper portions 38, within the scope of the present disclosure. The different arrangements can be selected by those skilled in the art to impart increased stiffness to different portions or sides of the supporting plate 12 and/or to provide additional support (i.e., resistance to deflection) for portions of the supporting plate 12 that are expected to be exposed to substantial forces during use.

In some examples, as shown in FIGS. 1A-1C, the holding device 10 includes the supporting plate 12 having surfaces 14, 16 comprising the flat peripheral lower portion 36, the upwardly angled portion 40 comprising a substantially vertical wall, and a single flat upper portion 38 over a center of the supporting plate 12. As shown in FIGS. 1A-1C, all of the inner openings 24 extend through the upper portion 38 of the surfaces 14, 16. Further, the height H1 (shown in FIG. 1C) of the upper portion 38 is substantially the same as the height of the chimneys 30. Also, the upwardly angled portion 40 is substantially parallel to the axes X1, X2 of the supporting plate 12 and does not include inwardly or outwardly angled segments.

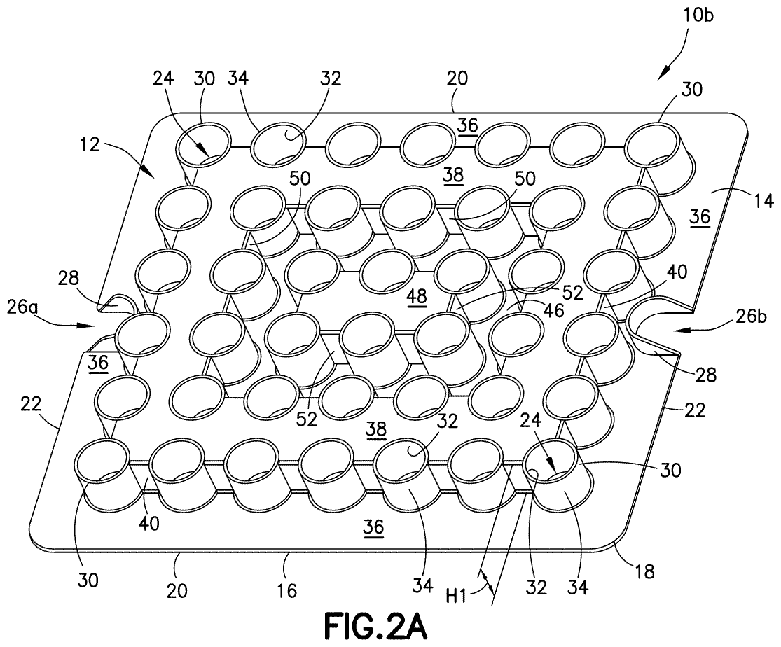

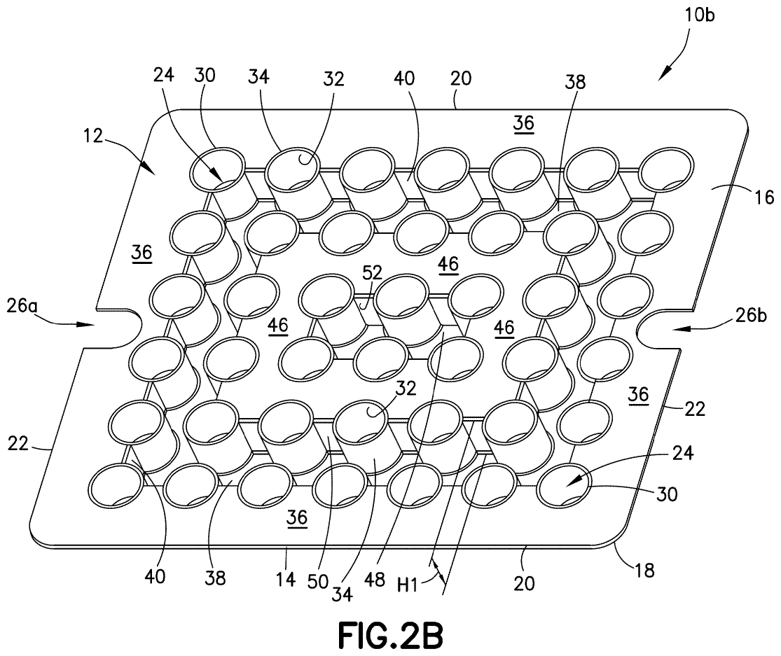

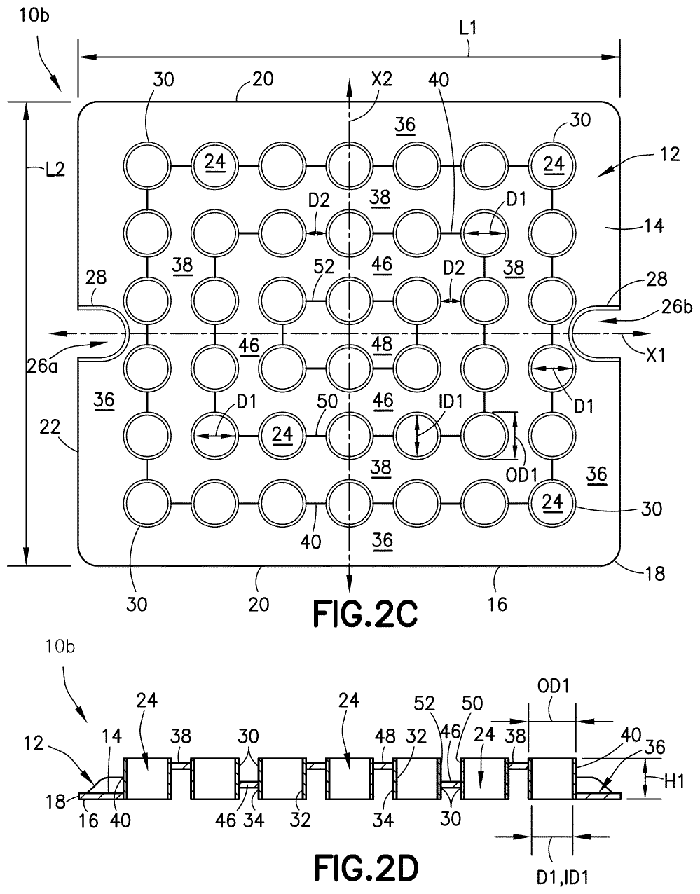

FIGS. 2A-2D illustrate another example of a holding device 10b including multiple lower portions and multiple upper portions. More specifically, as shown in FIGS. 2A-2D, the surfaces 14, 16 of the supporting plate 12 include the peripheral lower portion 36, the upwardly angled portion 40 comprising a substantially vertical wall, and the upper portion 38, as in previous examples. The surfaces 14, 16 of the supporting plate 12 also include a second or inner lower portion 46 and a second or inner upper portion 48 over a center of the supporting plate 12. The surfaces 14, 16 also include a downwardly angled portion 50 that extends between the upper portion 38 and the second or inner lower portion 46 and a second or inner upwardly angled portion 52 extending between the inner lower portion 46 and the inner upper portion 48.

As previously described, the lower portions 36, 46 are lowest areas of the supporting plate 12, meaning that the second or lower surfaces 16 of the lower portions 36, 46 are configured to be in face-to-face contact with a flat substrate if the holding device 10b were positioned on the flat substrate. As in previous examples, the upper portions 38, 48 are elevated relative to the lower portions 36, 46 by the height H1, which can be the same height as the chimneys 30. Further, the angled portions 40, 50, 52 can be substantially vertical walls extending between the lower portions 36, 46 and the upper portions 38, 48 of the surfaces 14, 16. As in previous examples, the angled portions 40, 50, 52 of the surfaces 14, 16 of the supporting plate 12 are all parallel to the axes X1, X2 of the supporting plate 12 and do not include inwardly or outwardly angled segments.

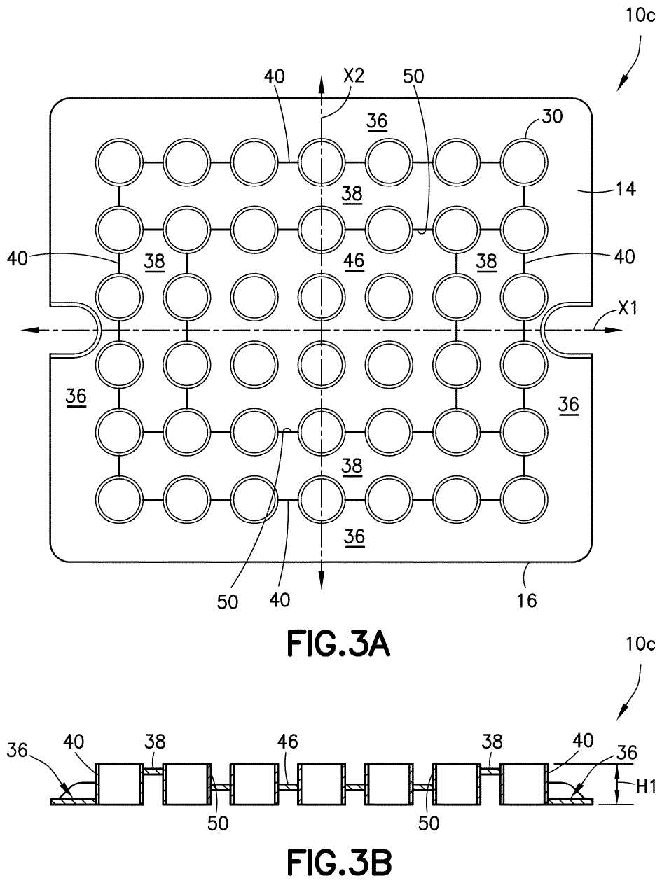

FIGS. 3A and 3B illustrate another example of a holding device 10c, which includes multiple lower portions and a single upper portion. More specifically, as shown in FIGS. 3A and 3B, the surfaces 14, 16 include the peripheral lower portion 36, the upper portion 38, and the upwardly angled portion 40 extending between the lower portion 36 and the upper portion 38, as in previous examples. The surfaces 14, 16 also include an inner lower portion 46 enclosed by the upper portion 38 and a downwardly angled portion 50 extending between the upper portion 38 and the inner lower portion 46. As in previous examples, the angled portions 40, 50 are substantially vertical walls that contact chimneys 30 of the supporting plate 12.

FIGS. 4A and 4B illustrate another exemplary holding device 10d including a segmented angled portion as previously described. More specifically, as shown in FIGS. 4A and 4B, the holding device 10d includes the supporting plate 12 including surfaces 14, 16. The surfaces 14, 16 include the peripheral lower portion 36, the upper portion 38, and the upwardly angled portion 40 extending between the peripheral lower portion 36 and the upper portion 38. As in previous examples, the upwardly angled portion 40 comprises a substantially vertical wall that passes through or is connected to outer chimneys 30 of the holding device 10d. Unlike in previous examples, the upwardly angled wall of FIGS. 4A and 4B includes angled segments or portions. Specifically, the portions of the upwardly angled wall 40 proximate to the transverse sides 22 of the supporting plate 12 are segmented including the alternating inwardly directed segments 42 and outwardly directed segments 44. As shown in FIG. 4B, the inwardly directed segments 42 are angled in a direction of arrow A1 towards the second or transverse axis X2 of the supporting plate 12. The outwardly directed segments 44 are angled, in a direction of arrow A2, away from the transverse or second axis X2 of the supporting plate 12. As shown in FIGS. 4A and 4B, the portions of the upwardly angled portion 40 proximate to the longitudinal sides 20 of the supporting plate 12 are straight or flat extending parallel to the longitudinal or first axis X1 of the supporting plate 12 and are not inwardly or outwardly angled.

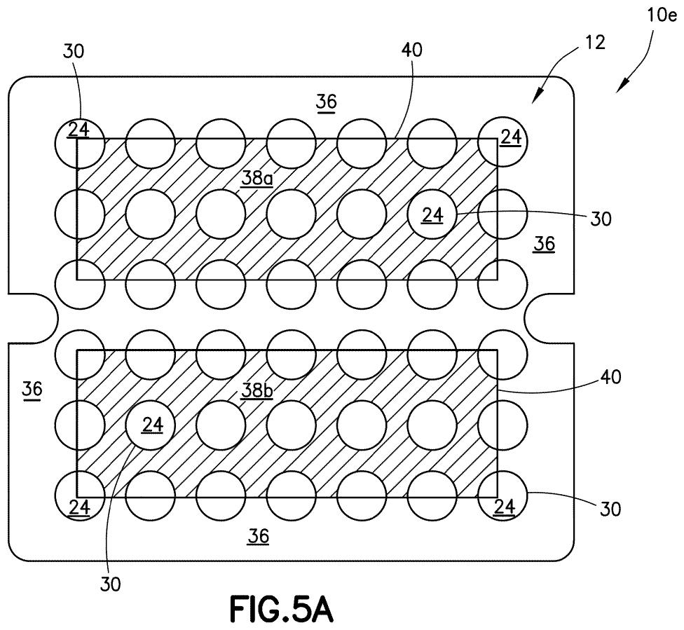

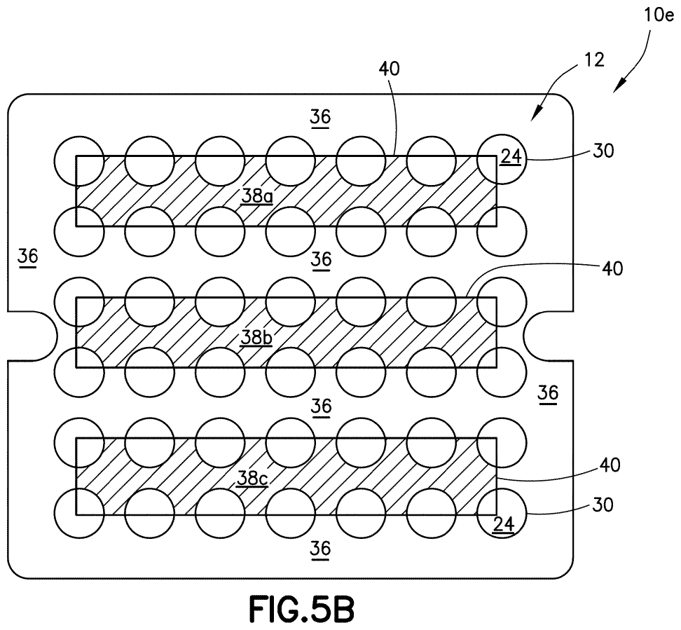

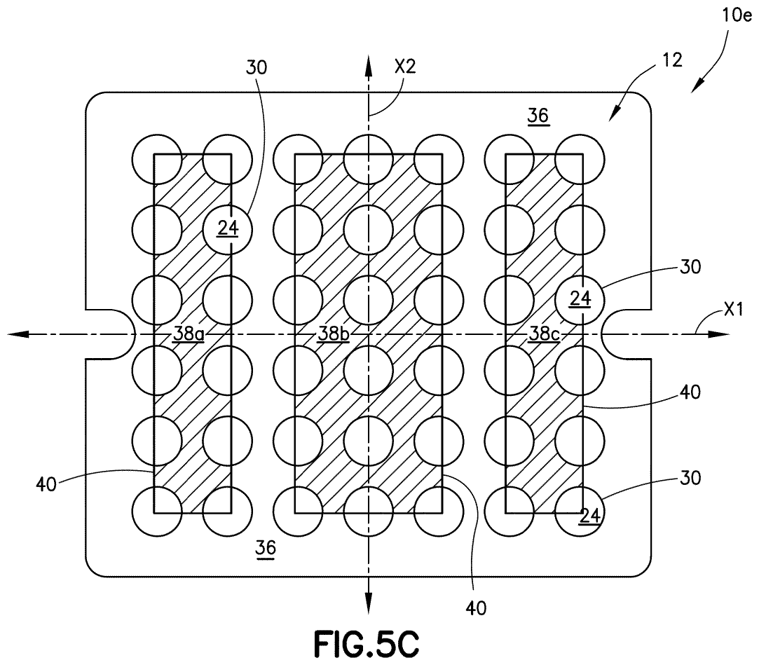

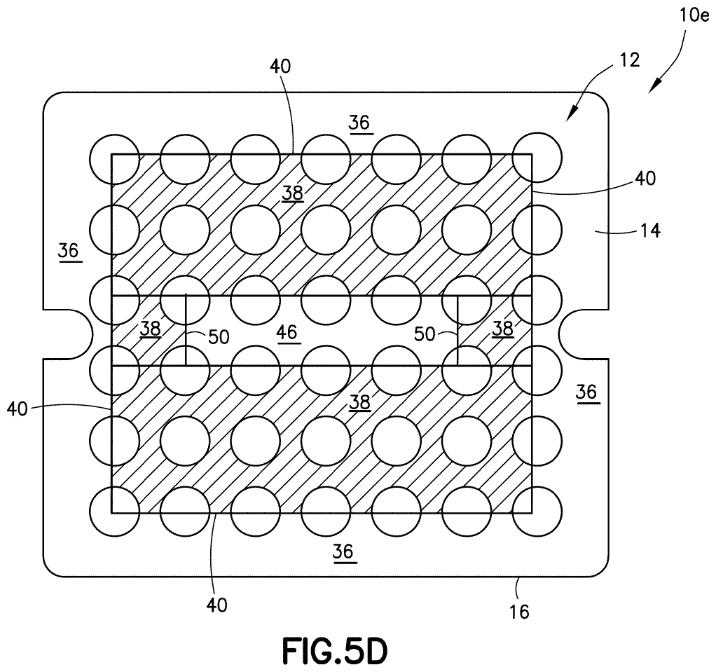

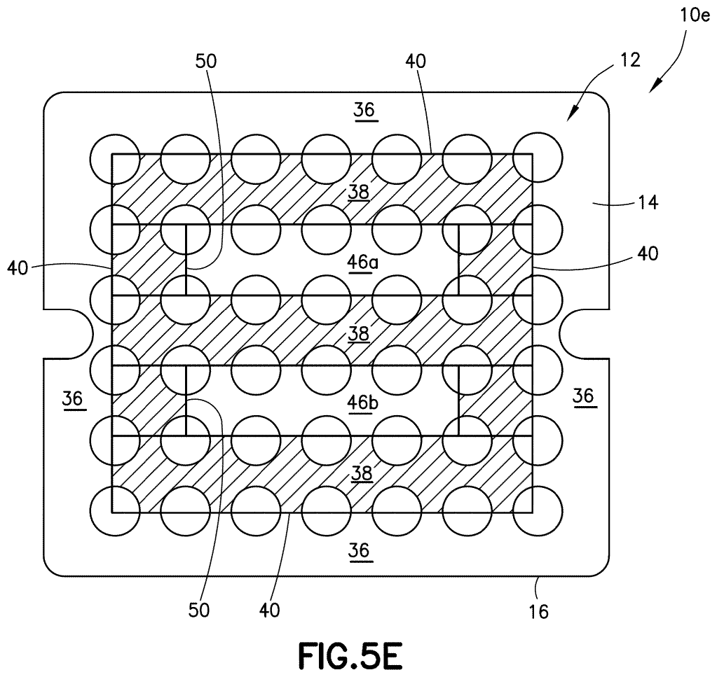

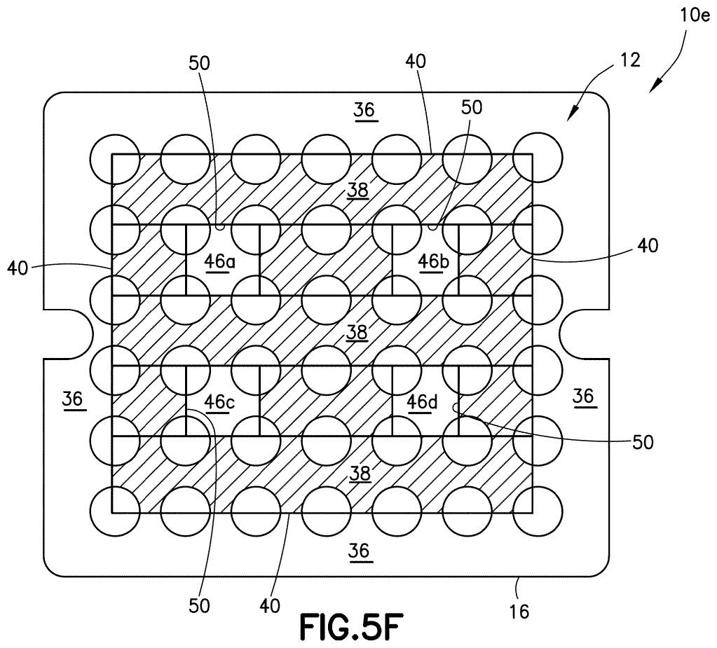

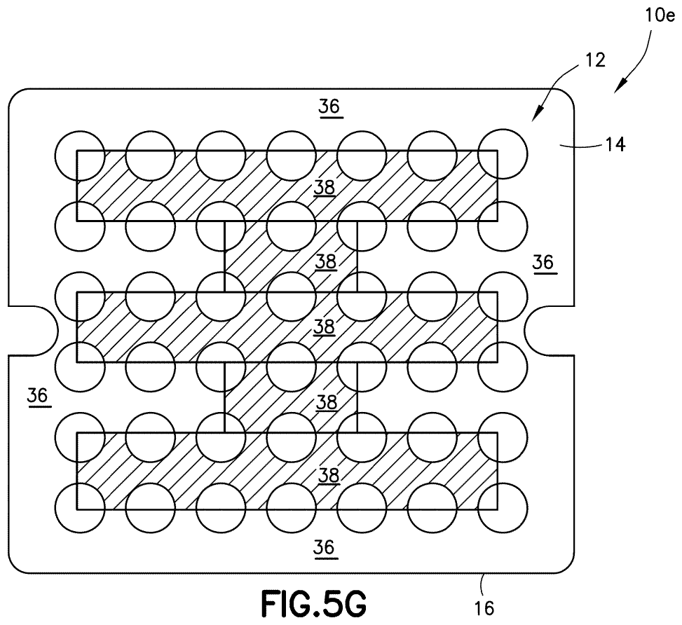

FIGS. 5A-5G show additional examples of holding devices 10e including different arrangements of lower portions 36, upper portions 38, 38a, 38b, and upwardly angled portions 40. In FIGS. 5A-5G, the upper portions 38, 38a, 38b are representing by cross-hatching. The upwardly angled portions 40 of FIGS. 5A-5G are straight or unsegmented portions that do not include the previously described inwardly or outwardly directed segments.

For example, FIG. 5A illustrates a holding device 10e including the peripheral lower portion 36 and two separate upper portions 38a, 38b enclosed by the lower portion 36. Each of the separate upper portions 38a, 38b is enclosed by a rectangular shaped upwardly angled portion 40 comprising a vertical wall. The upwardly angled portions 40 are connected to or pass through both outer chimneys 30 and inner chimneys 30 of the supporting plate 12. Similarly, FIG. 5B illustrates a holding device 10e including a peripheral lower portion 36 and three separate upper portions 38a, 38b, 38c enclosed by the lower portion 36. Each upper portion 38a, 38b, 38c is enclosed by a rectangular upwardly angled portion 40 comprising a vertical wall. FIG. 5C illustrates a similar holding device 10e including the peripheral lower portion 36 and three separate upper portions 38a, 38b, 38c. The three separate upper portions 38a, 38b, 38c are arranged in a transverse position, with a central axis of each of the rectangular upper portions 38a, 38b, 38c being parallel to the second or transverse axis X2 of the supporting plate 12.

FIG. 5D illustrates a holding device 10e including a supporting plate 12 with surfaces 14, 16 including the peripheral lower portion 36, the upper portion 38, and the upwardly angled portion 40 between the lower portion 36 and the upper portion 38. The surfaces 14, 16 also include an inner lower portion 46 that is enclosed by the upper portion 38. The surfaces 14, 16 also include a downwardly angled portion 50 that extends between the upper portion 48 and the inner lower portion 46. Similarly, FIGS. 5E and 5F illustrate holding devices 10e including the supporting plate 12 with surfaces 14, 16 including the peripheral lower portion 36, the upper portion 38, and the upwardly angled portion 40 between the lower portion 36 and the upper portion 38. The surfaces 14, 16 of FIGS. 5E and 5F also include multiple inner lower portions 46a, 46b, 46c, 46d that are enclosed by the upper portion 38. For example, the holding device 10e of FIG. 5E includes two inner lower portions 46a, 46b and the holding device 10e of FIG. 5F includes four inner lower portions 46a, 46b, 46c, 46d. The holding devices 10e of FIGS. 5E and 5F also include separate downwardly angled portions 50 that extend between the upper portion 38 and the separate inner lower portions 46a, 46b, 46c, 46d of the surfaces 14, 16 of each supporting plate 12.

FIG. 5G illustrates a holding device 10e including the peripheral lower portion 36 and an irregularly shaped upper portion 38. Specifically, the irregularly shaped upper portion 38 of FIG. 5G includes three long rectangles that are connected together by two short rectangles. The surfaces 14, 16 of the supporting plate 12 also include an irregularly-shaped upwardly angled portion 40 that extends around the irregular upper portion 38 and between the peripheral lower portion 36 and the upper portion 38 of the surfaces 14, 16.

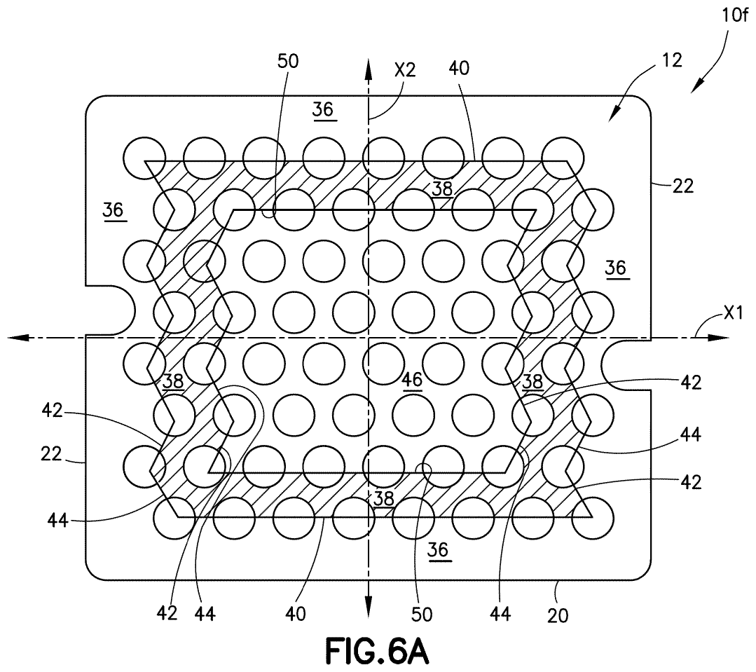

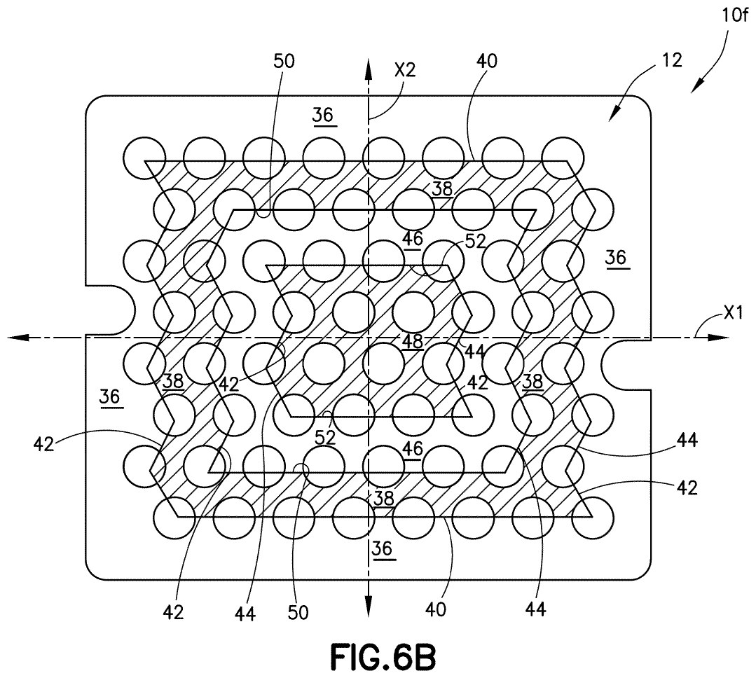

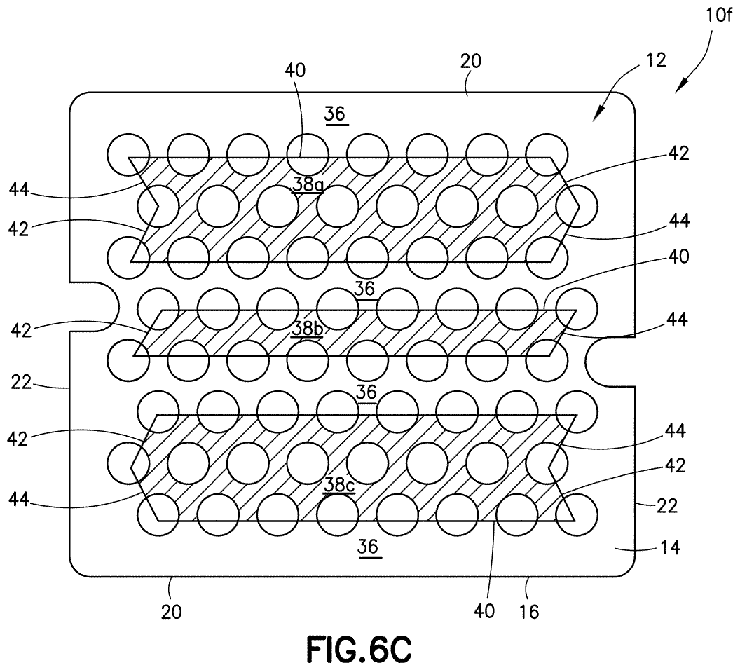

FIGS. 6A-6C illustrate holding devices 10f including different arrangements of peripheral lower portions 36, upper portions 38, 38a, 38b, 38c, and angled portions 40 that include inwardly directed segments 42 and/or outwardly directed segments 44. In FIGS. 6A-6C, the upper portions 38, 38a, 38b, 38c, as well as other raised surfaces such as the inner upper portion 48, are represented by cross-hatching. For example, as previously described, the segmented angled portions 40 can include alternating inwardly directed segments 42 that are angled towards one of the axes X1, X2 of the supporting plate 12 and outwardly directed segments 44 that are angled away from the axes X1, X2 of the supporting plate 12.

In some examples, as shown in FIG. 6A, the holding device 10f includes the peripheral lower portion 36, the upper portion 38, and a second or inner lower portion 46. The lower and upper portions 36, 38, 46 are connected by the segmented angled portions 40, 50. Specifically, as shown in FIG. 6A, the upwardly angled portion 40 extends between the peripheral lower portion 36 and the upper portion 38. Portions of the upwardly angled portion 40 proximate to the transverse sides 22 of the supporting plate 12 are segmented including the inwardly directed segments 42 and the outwardly directed segments 44. The upwardly angled portion 40 proximate to the longitudinal sides 20 of the supporting plate 12 are straight or flat extending substantially parallel to the longitudinal or first axis X1 of the supporting plate 12. The surfaces 14, 16 of the supporting plate 12 also include the downwardly angled portion 50 extending between the upper portion 38 and the second or inner lower portion 46. The downwardly angled portion 50 is segmented including the inwardly directed portions 42 and the outwardly directed portions 44 in the same manner as the previously described upwardly angled portion 40.

FIG. 6B illustrates a similar holding device 10f including the peripheral lower portion 36, the segmented upwardly angled portion 40, the upper portion 38, the segmented downwardly angled portion 50, and the second or inner lower portion 46. As shown in FIG. 6B, the surfaces 14, 16 also include a second or inner upper portion 48 and a segmented inner upwardly angled portion 52 extending between the inner lower portion 46 and the second or inner upper portion 48. The surfaces 14, 16 also include a second or inner upwardly angled portion 52 extending between the inner lower portion 46 and the inner upper portion 48. The second or inner upwardly angled portion 52 is segmented including the inwardly directed portions 42 and the outwardly directed portions 44 in the same manner as the previously described segmented angled portions.

FIG. 6C illustrates another exemplary holding device 10f including the peripheral lower portion 36 and three separate upper portions 38a, 38b, 38c. The surfaces 14, 16 of the supporting plate 12 also include separate upwardly angled portions 40 extending about each of the separate upper portions 38a, 38b, 38c. As in previous examples, portions of the upwardly angled surfaces 40 proximate to the transverse sides 22 of the supporting plate 12 are segmented including the inwardly directed segments or portions 42 and the outwardly directed segments or portions 44.

FIGS. 7A and 7B illustrate additional examples of holding devices 10g. The holding devices 10g of FIGS. 7A and 7B include the surfaces 14, 16 including the peripheral lower portion 36, an upwardly angled portion 40, and an upper portion 38. In FIG. 7A, the angled portion 40 is only slightly angled and does not comprise a vertical wall. By contrast, the angled portion 40 of FIG. 7B includes the vertical wall connected to or passing through the outer chimneys 30 of the holding device 10g. The upper portions 38 of the surfaces 14, 16 of the holding devices 10g of FIGS. 7A and 7B differ from previous examples because the upper portions 38 are curved or arcuate rather than flat. Specifically, as shown in FIG. 7A, the upper portion 38 of the surfaces 14, 16 is convex. In the holding device 10g of FIG. 7B, the upper portion 38 of the surfaces 14, 16 is concave.

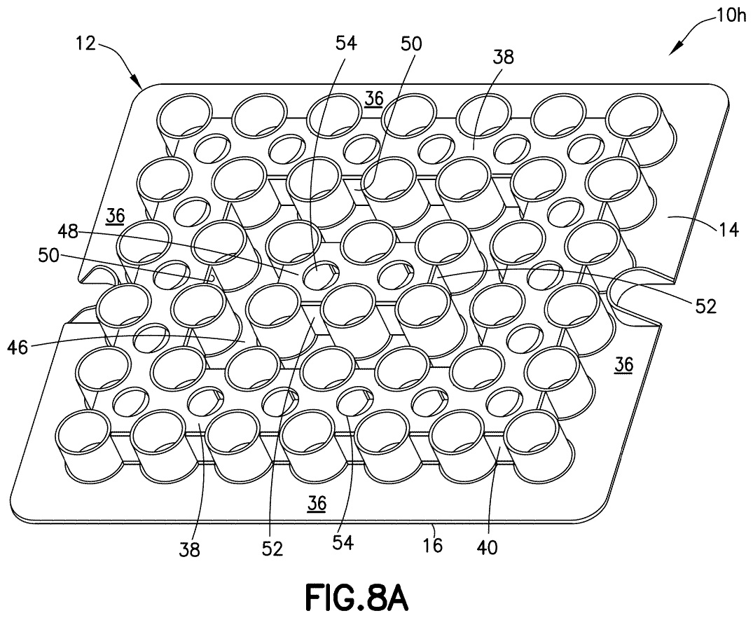

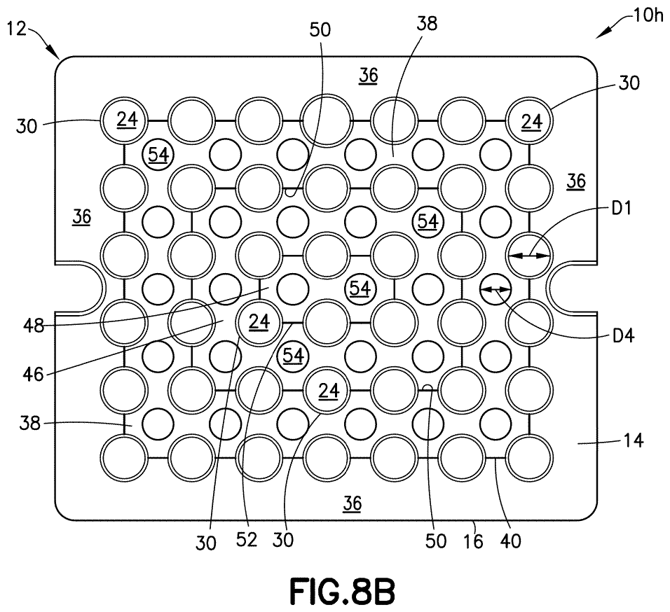

FIGS. 8A and 8B illustrate another example of a holding device 10h including multiple lower portions and multiple upper portions. The holding device 10h is similar to the holding device 10b shown in FIGS. 2A-2D, except that additional openings or holes are added to reduce a total weight and an amount of thermoplastic material needed to form the holding device 10h. While the holding device 10h may be less rigid than previous examples due to the lower total weight and less material, the holding device 10h is more rigid than conventional plates or trays that do not include surfaces of different elevations.

More specifically, as shown in FIGS. 8A and 8B, the surfaces 14, 16 of the supporting plate 12 include a peripheral lower portion 36, an upwardly angled portion 40 comprising a substantially vertical wall, and an upper portion 38. The surfaces 14, 16 of the supporting plate 12 also include a second or inner lower portion 46 and a second or inner upper portion 48 over a center of the supporting plate 12. The surfaces 14, 16 also include a downwardly angled portion 50 that extends between the upper portion 38 and the second or inner lower portion 46 and a second or inner upwardly angled portion 52 extending between the inner lower portion 46 and the inner upper portion 48.

The supporting plate 12 further includes the additional openings or holes 54 for reducing material of the holding device 10. The openings or holes 54 can be any of a variety of sizes or shapes including circles, ovals, squares, or rectangles, as well as elongated grooves or channels extending through the surfaces 14, 16 of the supporting plate 12. In some examples, the openings or holes 54 can be circles. The circles can have a diameter D4 that is smaller than the diameter DI of the openings 24 enclosed by the chimneys 30. The number and position of the openings or holes 54 can be selected by those skilled in the art by considering the importance of reducing the total weight of the holding device 10 along with the desired rigidity of the holding device 10. For example, as shown in FIGS. 8A and 8B, the supporting plate 12 can include thirty openings or holes 54. The openings or holes 54 can be positioned, for example, on the upper portions 38, 48 and/or lower inner portion 46 of the surfaces 14, 16 of the supporting plate 12. In some examples, the lower peripheral portion 36 of the surfaces 14, 16 is free from additional openings or holes 54.

The holding devices 10-10h of the present disclosure can be made of various plastic materials commonly used for disposable medical packaging, containers, and/or devices. Further, the holding devices 10-10h can be made by various forming and molding processes, as are known in the art. For example, the holding devices 10-10h of the present disclosure can comprise a thermoplastic material, preferably polypropylene, or polyester, polycarbonate, polyethylene, polyethylene terephthalate, acrylonitrile butadiene styrene, or other injection moldable or formable resin materials, as are known in the art. In some examples, the supporting plate 12 and chimneys 30 can be integrally formed from a same material, such as from the same thermoplastic material. In some examples, the holding devices 10-10h are formed by a single injection molding process, in which the holding device 10-10h is formed by injecting a fluid polymer material between a first tool and a second tool in a single process. In order to optimize the holding devices 10-10h for injection molding, in some examples, vertical or substantially vertical surfaces of the holding device 10-10h, such as the previously described upwardly or downwardly angled surfaces 40, 50, 52 comprising vertical walls, as well as the inner surface 32 and/or the outer surface 34 of the chimneys 30, can include a slightly sloped surface having a draft angle (e.g., an angle of about 0.1 degree to 4.0 degrees, for example, 1.0 degree or 0.5 degree). Including substantially vertical surfaces with a slight slope or draft angle makes the molded part easier to remove from the mold following injection molding. In other examples, the holding devices 10-10h can be made by three-dimensional printing by processes known in the art.

While examples of the holding devices 10-10h are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A holding device configured to support medical containers, comprising:
a supporting plate comprising a first surface, a second surface, a peripheral edge between the first surface and the second surface, and a plurality of openings through the supporting plate configured to receive the medical containers, wherein the surfaces of the supporting plate comprise:
a peripheral lower portion extending inward from the peripheral edge;
at least one upper portion enclosed by the peripheral lower portion; and
at least one upwardly angled portion extending between the lower portion and the upper portion; and
a plurality of chimneys protruding from the first surface and/or the second surface of the supporting plate at least partially enclosing the plurality of openings, such that the plurality of chimneys guide insertion of the medical containers into the plurality of openings.

2. The holding device of claim 1, wherein the plurality of openings are arranged on the supporting plate as a plurality of rows and a plurality of columns.

3. The holding device of claim 1, wherein openings of adjacent rows are aligned parallel to a transverse axis of the supporting plate and openings of adjacent columns are aligned parallel to a longitudinal axis of the supporting plate orthogonal to the transverse axis.

4. The holding device of claim 1, wherein, when fully loaded with the medical containers, deflection of the supporting plate is less than 2.5 mm in a vertical direction.

5. The holding device of claim 1, wherein the supporting plate and the plurality of chimneys are integrally formed and is made from a thermoplastic polymer.

6. The holding device of claim 1, wherein the supporting plate comprises fewer than 100 openings.

7. The holding device of any of claim 1, wherein the peripheral lower portion, the upper portion, and the angled portion each intersect chimneys of the plurality of chimneys.

8. The holding device of claim 1, wherein the peripheral lower portion and the upper portion of the surfaces are flat.

9. The holding device of claim 1, wherein the upper portion is elevated relative to the lower portion by a vertical distance of about 2.0 mm to about 14.5 mm.

10. The holding device of claim 1, wherein the at least one upper portion of the surfaces is a convex surface or a concave surface.

11. The holding device of claim 1, wherein the angled portion is substantially vertical extending substantially orthogonally from the lower portion, and having a draft angle of about 0.1 degree to about 4.0 degrees.

12. The holding device of claim 1, wherein the angled portion comprises alternating inwardly angled segments, which are angled towards a center of the first surface and/or the second surface of the supporting plate, and outwardly angled segments, which are angled away from the center of the first surface and/or the second surface of the supporting plate.

13. The holding device of claim 1, wherein the plurality of chimneys comprise a tubular wall comprising a cylindrical inner surface having a diameter substantially the same as the diameter of the opening enclosed by the chimney and a cylindrical outer surface opposite the inner surface.

14. The holding device of claim 1, wherein the plurality of openings and the plurality of chimneys are configured to receive barrels of syringes or cartridges containing a fluid volume of less than 50 mL.

15. The holding device of claim 1, wherein the surfaces of the supporting plate further comprise a second lower portion at least partially enclosed by the upper portion and a downwardly angled portion extending between the upper portion and the second lower portion.

16. The holding device of claim 1, wherein the at least one upper portion comprises a first upper portion and a second upper portion, which is separated from the first upper portion by the lower portion.

17. The holding device of claim 1, wherein the first upper portion and the second upper portion each intersect at least one of the plurality of chimneys.

18. The holding device of claim 1, wherein the surfaces of the supporting plate further comprise a second upper portion at least partially enclosed by the second lower portion and a second upwardly angled surface extending between the second lower portion and the second upper portion.

19. An assembly configured to support a plurality of medical containers, the assembly comprising:
a tub; and
the holding device of claim 1 contained in the tub.

20. A method of manufacture for the holding device of claim 1, the method comprising injection molding or three-dimensional printing the support plate and the plurality of chimneys by a single injection molding or three-dimensional printing process.
